(19) United States
(12) Patent Application Publication
(10) Pub. No.: US 2007/0196292 A1
(43) Pub. Date: Aug. 23, 2007

(54) Personal care composition comprising dehydroacetate salts

(76) Inventors: Larry Richard Robinson, Loveland, OH (US); Yumo Zhang, Algonquin, IL (US)

Correspondence Address: THE PROCTER & GAMBLE COMPANY;INTELLECTUAL PROPERTY DIVISION - WEST BLDG., WINTON HILL BUSINESS CENTER - BOX 412, 6250 CENTER HILL AVENUE, CINCINNATI, OH, 45224, US

(21) Appl. No.: 11/593,412

(22) Filed: Nov. 6, 2006

Related U.S. Application Data
(60) Provisional application No. 60/741,363, filed on Nov. 30, 2005.

(51) Int. Cl. A61K 8/36; A61K 31/59; A61K 31/19; A61K 8/97; A61K 36/82

(52) U.S. Cl. 424/59; 424/401; 424/74; 424/62; 514/557; 514/167; 424/729

(57) ABSTRACT

Personal care composition comprising a dehydroacetate salt, and a dermatologically acceptable carrier, wherein the pH of said composition is about 7.0 and greater.

### CROSS REFERENCE TO RELATED APPLICATION

This application claims the benefit of U.S. Provisional Application No. 60/741,363, filed Nov. 30, 2006.

### FIELD OF THE INVENTION

The present invention relates to personal care composition comprising dehydroacetate salts, suitable for improving the appearance and condition of mammalian keratinous tissue.

### BACKGROUND OF THE INVENTION

Maintaining the health and appearance of skin and other keratinous tissue is important to many consumers. In particular, there is considerable interest in avoiding and minimizing what many consider undesirable “signs of skin aging,” for example, fine lines, wrinkles and uneven skin texture. Personal care compositions often contain one or more active ingredients to help minimize these undesired effects. Dehydroacetic acid has proven effective in improving the texture and appearance of keratinous tissue, and is a desirable component in personal care compositions. The use of dehydroacetic acid significantly lowers the pH of personal care compositions. Applicants have found that under low-pH, or acidic, conditions, dehydroacetic acid is subject to instability, and may cause compositions to exhibit discoloration, which many consumers find unacceptable. A need exists, therefore, to develop a personal care composition that can provide to keratinous tissue the benefits of dehydroacetic acid, and which exhibit minimal discoloration.

### SUMMARY OF THE INVENTION

The present invention meets the aforementioned need. At a basic pH, dehydroacetic acid is essentially entirely in the form of a dehydroacetate salt, which provides similar benefits to keratinous tissue as dehydroacetic acid. Applicants have found that in compositions with a pH of 7.0 and greater, the concentration of the dehydroacetate salt is optimized. Additional advantages include minimization of discoloration of the composition, and that a composition at a pH of 7.0 or above is more closely aligned with the natural pH of the skin, which typically ranges from slightly acidic to slightly basic. Thus, the composition may be more compatible with the chemical characteristics of the skin, and more readily absorbed. The present invention therefore meets the need of providing a composition that provides the benefits of dehydroacetic acid, and which exhibit acceptable minimal discoloration and good chemical stability.

The following represent non-limiting embodiments of the present invention.

According to the first embodiment of the present invention, a personal care composition is provided for regulating the condition of mammalian keratinous tissue. The composition comprises a dehydroacetate salt and a dermatologically acceptable carrier. The pH of the composition is at least 7.0. In an alternative embodiment, the composition comprises at least one additional skin care active.

Yet another embodiment provides for depositing a personal care composition according to the first embodiment onto a substrate, such as a wipe.

Yet another embodiment provides a method for regulating the condition of mammalian keratinous tissue. The method comprises the step of applying to the mammalian skin a stable personal care composition comprising a dehydroacetate salt in a dermatologically acceptable carrier, wherein the pH of said composition is at least 7.0.

According to yet another embodiment of the present invention, a kit is provided for regulating the condition of mammalian keratinous tissue, comprising a composition according to the first embodiment of the present invention.

### DETAILED DESCRIPTION OF THE INVENTION

Whereas the specification concludes with claims that particularly point out and distinctly claim the present invention, it is believed that the invention will be better understood from the following details.

The present invention describes a personal care composition comprising a dehydroacetate salt and a dermatologically acceptable carrier. Applicants have found that at a pH of at least 7.0, the stability of the dehydroacetate salt is optimized, and the composition exhibits minimal discoloration. The pH of the composition of the present invention therefore is at least 7.0.

The composition of the present invention may take a variety of final forms, non-limiting examples of which include a lotion, cream, emulsion, paste, milk, liquid, gel, solid, spray, mousse, eye jelly, mask, and combinations thereof. The composition may be applied to the skin via a variety of means, and may be used in combination with a delivery enhancement device, non-limiting examples of which include an implement, a spray applicator, a brush, an automated scrubbing device, and combinations thereof. The composition of the present invention optionally may include additional skin care actives useful for regulating the condition of mammalian keratinous tissue, conditioning agents, emollients, etc. The composition further may be releasably applied to a carrier substrate, suitable for use at a later time. The composition further may be used in conjunction with orally ingestible dietary supplement to provide enhanced skin care benefits.

The present invention includes both compositions that are intended to be left on the keratinous tissue indefinitely, or “leave-on” compositions, and compositions which are intended to be removed from the keratinous tissue. Removal may occur through a variety of means, for example wiping or rinsing with water. The rinse-off composition may be in the form of a liquid, or also may be in the form of a lotion, or “cleansing milk.”

In addition to dehydroacetate salts, the composition of the present invention optionally may contain other skin care actives that are reasonably stable in basic pH conditions.

Each of the above and additional elements is described herein.

In all embodiments of the present invention, all percentages are by weight of the total composition, unless specifically stated otherwise. All ratios are weight ratios, unless specifically stated otherwise. The number of significant digits conveys neither limitations on the indicated amounts nor on the accuracy of the measurements. All amounts indicating quantities, percentages, proportions and pH measurements are understood to be modified by the word “about” unless otherwise specifically indicated. All measurements are understood to be made at 25° C. and at ambient conditions, where “ambient conditions” means conditions under about one atmosphere of pressure and at about 50% relative humidity.

Herein, “high pH,” or alternatively “basic pH,” means a pH of about 7.0 and greater. All pH measurements are made by standard means that would be known to one skilled in the art. The term “pH of the composition,” or other language describing the pH of the composition means the pH of the undiluted, neat composition, measured after the composition is cooled to 25° C., unless otherwise indicated. The pH of the composition of the present invention is about 7.0 and greater. Alternatively, the pH is from about 7.0 to about 11.0. Alternatively, the pH is from about 7.0 to about 9.0.

Herein, “stable” and “stability” mean a composition which is substantially unaltered in chemical state, physical homogeneity and/or color upon exposure to conditions reasonably expected to be incurred in shipping, storage and use. Stability may be determined either by empirical observation or by appropriate methods of chemical and/or physical analysis that would be known to one of skill in the art.

“Keratinous tissue,” as used herein, means keratin-containing layers disposed as the outermost protective covering of mammals and includes, but is not limited to, skin, hair and nails. “Topical application,” as used herein, means to apply or spread a composition onto the surface of the keratinous tissue.

Herein, “personal care composition” means a composition suitable for topical application on mammalian keratinous tissue. The personal care composition described herein may contain one or more skin care actives. “Skin care actives,” or “actives,” as used herein, means compounds that aid in regulating the condition of skin and of other mammalian keratinous tissue, for example, by providing a benefit or improvement to the keratinous tissue.

Herein, “regulating the condition of keratinous tissue” means improving the condition of mammalian keratinous tissue and/or prophylactically regulating the condition of mammalian keratinous tissue, and includes, for example, protecting the tissue from ultraviolet radiation, and regulating the signs of skin aging. Herein, “improving the condition of mammalian keratinous tissue” means effecting a visually and/or tactilely perceptible positive change in the appearance and feel of the tissue. Conditions that may be regulated and/or improved include, but are not limited to, one or more of the following: Reducing the appearance of wrinkles and coarse deep lines, fine lines, crevices, bumps, and large pores; thickening of keratinous tissue (e.g., building the epidermis and/or dermis and/or sub-dermal layers of the skin, and where applicable the keratinous layers of the nail and hair shaft, to reduce skin, hair, or nail atrophy); increasing the convolution of the dermal-epidermal border (also known as the rete ridges); preventing loss of skin or hair elasticity, for example, due to loss, damage and/or inactivation of functional skin elastin, resulting in such conditions as elastosis, sagging, loss of skin or hair recoil from deformation; reduction in cellulite; change in coloration to the skin, hair, or nails, for example, under-eye circles, blotchiness (e.g., uneven red coloration due to, for example, rosacea), sallowness, discoloration caused by telangiectasia or spider vessels, dryness, brittleness, and graying hair.

As used herein, “signs of skin aging,” include, but are not limited to, outward visibly and tactilely perceptible manifestations, as well as any macro- or microeffects, due to keratinous tissue aging. These signs may result from processes which include, but are not limited to, the development of textural discontinuities such as wrinkles and coarse deep wrinkles, fine lines, skin lines, crevices, bumps, large pores, unevenness or roughness; flaking; dryness; loss of skin elasticity; discoloration (including undereye circles); blotchiness; sallowness; hyperpigmented skin regions such as age spots and freckles; keratoses; abnormal differentiation; hyperkeratinization; elastosis; collagen breakdown, and other histological changes in the stratum corneum, dermis, epidermis, vascular system (e.g., telangiectasia or spider vessels), and underlying tissues (e.g., fat and/or muscle), especially those proximate to the skin.

“Dermatologically-acceptable,” as used herein, means that the composition or components thereof so described are suitable for use in contact with mammalian keratinous tissue without undue toxicity, incompatibility, instability, allergic response, and the like.

Herein, “orally acceptable” means that the composition or components thereof so described are suitable for oral ingestion by a mammal without undue toxicity, incompatibility, instability, allergic response, etc.

“Effective amount,” as used herein, means an amount of a compound or composition sufficient to significantly induce a positive benefit, including independently or in combination the benefits disclosed herein, but low enough to avoid serious side effects.

Herein, “dietary supplement” means a composition comprising dietary ingredient intended to supplement a regular diet, non-limiting examples of which include vitamins, minerals, herbs or other botanicals, amino acids, enzymes and metabolites. The form in which the dietary supplement is administered may vary widely, and includes, for example, tablets, capsules, gel tablets, and liquids. The dietary supplement further may be incorporated into a foodstuff or beverage.

Herein, “delivery enhancement device” means any device that increases the amount of composition applied to and/or into the skin, more easily and/or efficiently delivers the composition, and/or increases the beneficial results derived from the composition, relative to that delivered without using the device. Examples of suitable delivery enhancement devices include, but are not limited to, implements such as a cotton ball, swab, pad, sponge, sponge-tipped applicator, spray applicator, brush, and combinations thereof.

Herein, “energy delivery device” means any device used to deliver energy to the skin, hair and other keratinous tissue. Examples of suitable energy delivery devices include, but are not limited to, ultrasonic devices, temperature change devices, heat delivery devices, radiofrequency wave devices, and combinations thereof.

Herein “kit” means a packaging unit comprising at least one composition described herein. The kit may comprise an outer packaging unit, which in turn may comprise one or more inner packaging units. The inner and outer packaging units may be of any type suitable for containing, presenting and/or reasonably protecting from damage the contents of the kit. The kit may comprise a plurality of components, including, but not limited to one or more orally ingestible dietary supplements, a delivery enhancement device, an implement, instructions for use of the device, instructions for complying with application regimens, and combinations thereof.

I. Dehydroacetate Salt

The composition of the present invention comprises a dehydroacetate salt. Herein, “dehydroacetate salt,” means the following compound, its isomers, derivatives and tautomers:

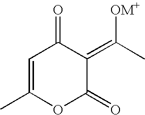

“M+” is a cationic species selected from the group consisting of Li⁺, Na⁺, K⁺, heavy metal salts that do not exhibit undue toxicity, trialkylammonium salts, such as those derived from trimethylamine, triethylamine, diethanolamine, triethanolamine, dialkylammonium salts, and mixtures thereof. Herein, the cationic species determines the type of dehydroacetate salt. For example, when M⁺ is sodium, the dehydroacetate salt is referred to as sodium dehydroacetate. In one embodiment, the dehydroacetate salt is selected from the group consisting of sodium dehydroacetate, potassium dehydroacetate, triethanolamine dehydroacetate and mixtures thereof. Alternatively, the dehydroacetate salt is sodium dehydroacetate.

One technical name for the sodium dehydroacetate salt of the present invention is 3-acetyl-6-methyl-2H-pyran-2,4,(3H)-dione, Ion (1-), sodium salt. This compound can be made, for example, by adding a sufficient amount of sodium hydroxide to dehydroacetic acid, which can be commercially purchased from Tri-K Industries (Northvale, N.J.), and under the tradename GEOGARD® 221 or GEOGARD® 361 from Lonza (Annandale, N.J.). In one embodiment, the composition comprises from about 0.001% to about 10% of a dehydroacetate salt. Alternatively, the composition includes from about 0.01% to about 5% of a dehydroacetate salt. Alternatively, the composition includes from about 0.1% to about 1% of a dehydroacetate salt.

Derivatives of dehydroacetate salts include, but are not limited to, any compounds wherein one or more of the hydrogen atoms of the CH₃ groups are individually or in combination replaced by amides, esters, amino groups, alkyls, and alcohol esters. Tautomers of dehydroacetate salts are the isomers of dehydroacetate salts which can change into one another with ease so that they ordinarily exist in equilibrium. Thus, tautomers of dehydroacetate salts can be described as having the chemical formula C₈H₇O₄M⁺ and generally having the structure above.

II. Dermatologically Acceptible Carrier

The composition of the present invention also comprises a dermatologically acceptable carrier. Herein, the phrase “dermatologically acceptable carrier” means that the carrier is suitable for topical application to the keratinous tissue, has good aesthetic properties, is compatible with the actives of the present invention and any other components, and will not cause any safety or toxicity concerns. The composition of the present invention comprises from about 50% to about 99.99% of the dermatologically acceptable carrier, alternatively from about 60% to about 99.9% of the carrier, alternatively from about 70% to about 98% of the carrier, and alternatively from about 80% to about 95% of the carrier.

The dermatologically acceptable carrier can be in a wide variety of forms. Non-limiting examples include simple solutions (water-based or oil-based), solid forms (for example, gels or sticks) and emulsions. In one embodiment, the composition is in the form of an emulsion. Herein, “emulsions” generally contain an aqueous phase and a lipid or oil, and may contain a humectant, for example, glycerin. Lipids and oils may be derived from animals, plants, or petroleum and may be natural or synthetic. Emulsion carriers include, but are not limited to, oil-in-water, water-in-oil, water-in-silicone, silicone-in-water, water-in-oil-in-water, and oil-in-water-in-silicone emulsions. In one embodiment, the dermatologically acceptable carrier comprises oil-in-water emulsions and water-in-oil emulsions. In yet another embodiment, the dermatologically acceptable carrier is an oil-in-water emulsion.

Emulsifier

The composition of the present invention may comprise an emulsifier. Emulsifiers may be nonionic, anionic or cationic. Suitable emulsifiers are disclosed in, for example, U.S. Pat. No. 3,755,560 issued to Dickert et al., U.S. Pat. No. 4,421,769, issued to Dixon et al., and McCutcheon's Detergents and Emulsifiers, North American Edition, pages 317-324 (1986). Suitable emulsions may have a wide range of viscosities, depending on the desired product form.

III. Optional Ingredients

A. Skin Care Actives

In addition to dehydroacetate salts, the composition of the present invention optionally may contain at least one additional skin care active that exhibits stability at a pH of 7.0 or above. Classes of suitable skin care actives include, but are not limited to vitamins, including oil soluble vitamin B₃ derivatives (e.g., tocopheryl nicotinate) and retinoids, peptides and peptide derivatives, sugar amines, sunscreens and UV-absorbers, antioxidants, non-vitamin antioxidant radical scavengers, desquamation actives, chelating agents, anti-cellulite agents, topical anesthetics, sunless tanning agents, antimicrobial and/or antifungal actives, preservatives, and mixtures thereof. It should be noted, however, that many skin care actives may provide more than one benefit, or operate via more than one mode of action. Therefore, classifications herein are made for the sake of convenience and are not intended to limit the active to that particular application or applications listed.

1. Vitamins

The composition of the present invention may comprise one or more vitamins and pro-vitamins, their salts, isomers and derivatives. Non-limiting examples of suitable vitamins include: vitamin B compounds (including nicotinic acid, C1-C18 nicotinic acid esters (e.g., toccopheryl nicotinate and nicotinyl alcohol); B6 compounds, such as pyroxidine; and B5 compounds, such as panthenol, or “pro-B5”); retinoids, including vitamin A compounds and all natural and/or synthetic analogs of Vitamin A or retinol-like compounds which possess the biological activity of Vitamin A; vitamin E compounds, or tocopherol, including tocopherol sorbate, tocopherol acetate, other esters of tocopherol; vitamin C compounds, including ascorbyl esters of fatty acids, and ascorbic acid derivatives, for example, ascorbyl glucoside, magnesium ascorbyl phosphate, sodium ascorbyl phosphate, ascorbyl sorbate; vitamin D compounds; vitamin K compounds; and mixtures thereof. In one embodiment, the composition of the instant invention may comprise from about 0.0001% to about 20%, alternatively from about 0.001% to about 10%, alternatively from about 0.01% to about 5%, and alternatively from about 0.1% to about 1%, of the vitamin compound.

In one embodiment, the vitamin is selected from the group consisting of vitamin B compounds, vitamin C compounds, vitamin D compounds, and mixtures thereof. Alternatively, the vitamin is a vitamin C compound. Alternatively, the vitamin is magnesium ascorbyl phosphate.

2. Peptides and Peptide Derivatives

The composition of the present invention can comprise one or more peptides. Herein, “peptide” refers to peptides containing ten or fewer amino acids, their derivatives, including n-acyl derivatives, isomers, and complexes with other species such as metal ions (for example, copper, zinc, manganese, and magnesium). As used herein, peptide refers to both naturally occurring and synthesized peptides. In one embodiment, the peptides are di-, tri-, tetra-, penta-, and hexa-peptides, their salts, isomers, derivatives, and mixtures thereof. Examples of useful peptide derivatives include, but are not limited to, peptides derived from soy proteins, palmitoyl-lysine-threonine (pal-KT) and palmitoyl-lysine-threonine-threonine-lysine-serine (pal-KTTKS, or palmitoyl pentapeptide, available in a composition known as MATRIXYL®), palmitoyl-glycine-glutamine-proline-arginine (pal-GQPR, available in a composition known as RIGIN®), these three being available from Sederma, France, and Cu-histidine-glycine-glycine (Cu-HGG, also known as IAMIN®).

The composition may comprise from about 1×10⁻⁷% to about 20%, alternatively from about 1×10⁻⁶% to about 10%, and alternatively from about 1×10⁻⁵% to about 5% of the peptide.

3. Sugar Amines

The composition of the present invention may comprise a sugar amine, also known as amino sugars, and their salts, isomers, tautomers and derivatives. Sugar amine compounds useful in the present invention include, for example, N-acetyl-D-glucosamine, and also those described in PCT Publication No. WO 02/076423 and U.S. Pat. No. 6,159,485, issued to Yu, et al. In one embodiment, the composition comprises from about 0.01% to about 15%, alternatively from about 0.1% to about 10%, and alternatively from about 0.5% to about 5%, of the sugar amine.

4. Sunscreens and Ultraviolet Light Absorbers

The composition of the present invention may comprise one or more sunscreen actives and/or ultraviolet (UV) light absorbers. Herein, “sunscreen” is understood to include both sunscreen actives and UV light absorbers. The sunscreen may be organic or inorganic, and may be water-soluble, oil-soluble, a particulate material which is insoluble in either an oil or an aqueous phase, and mixtures thereof. In one embodiment the composition of the present invention comprises a water-soluble and an oil-soluble sunscreen. In one embodiment, the composition may comprise from about 1% to about 30%, and alternatively from about 2% to about 20% by weight of the composition, of the sunscreen. Exact amounts will vary depending upon the chosen sunscreen and the desired Sun Protection Factor (SPF) and spectrum of protection (e.g., UV-A and/or UV-B), and are within the knowledge and judgment of one of skill in the art.

Examples of suitable sunscreens are disclosed in The Cosmetic, Toiletry, and Fragrance Association's The International Cosmetic Ingredient Dictionary and Handbook, 10^{th} Ed., Gottschalck, T. E. and McEwen, Jr., Eds. (2004), p. 2267 and pp. 2292-93. Particularly suitable sunscreen actives include benzophenone-3, 3-benzylidene camphor, benzylidene camphor sulfonic acid, bis-ethylhexyloxyphenol methoxyphenyl triazine, butyl methoxydibenzoyl-methane, di-t-butyl hydroxy-benzylidene camphor, diethylhexyl butamido triazone, ethylhexyl dimethyl PABA, ethylhexyl methoxy-cinnamate, ethylhexyl methoxydibenzoyl-methane, ethylhexyl salicylate, ethylhexyl triazone, ethyl methoxycinnamate, homosalate, isoamyl cinnamate, isoamyl p-methoxycinnamate, isopropyl dibenzoylmethane, menthyl anthranilate, menthyl salicylate, 4-methylbenzylidene camphor, octocrylene, octrizole, PABA, PEG-25 PABA, phenylbenzimidazole sulfonic acid, polyacrylamidomethyl benzylidene camphor, potassium methoxy-cinnamate, potassium phenyl-benzimidazole sulfonate, TEA-salicylate, terephthalylidene dicamphor sulfonic acid, titanium dioxide, zinc oxide, and mixtures thereof.

5. Desquamation Actives

The composition of the present invention may comprise a desquamation active to enhance the appearance of the keratinous tissue. In one embodiment, the composition comprises from about 0.01% to about 10%, alternatively from about 0.5% to about 5%, and alternatively from about 0.1% to about 2% of a desquamation active. Non-limiting examples of suitable desquamation actives include sulfhydryl compounds and zwitterionic surfactants, described in U.S. Pat. No. 5,681,852, issued to Bissett; salicylic acid and zwitterionic surfactants, described in U.S. Pat. No. 5,652,228, issued to Bissett. In one embodiment, the desquamation active is cetyl betaine.

6. Skin Lightening Agents

The composition of the present invention may comprise a skin lightening agent. When used, the composition preferably comprise from about 0.1% to about 10%, more preferably from about 0.2% to about 5%, also preferably from about 0.5% to about 2%, by weight of the composition, of a skin lightening agent. Suitable skin lightening agents include those known in the art, including kojic acid, arbutin, tranexamic acid, ascorbic acid and derivatives, e.g., magnesium ascorbyl phosphate or sodium ascorbyl phosphate or other salts of ascorbyl phosphate, ascorbyl glucoside, and the like. Other skin lightening materials suitable for use herein include undecylenoyl phenylalanine (Sepiwhite® from SEPPIC), aloesin, Actiwhite® (Cognis), Emblica® (Rona, and Azeloglicina (Sinerga). In one embodiment, the skin lightening agent is an ascorbic acid derivative.

7. Antimicrobial and Antifungal Actives

The composition of the present invention may comprise an antimicrobial and/or antifungal active, for example, to destroy microbes, prevent microbe development, and/or to prevent the pathogenic action of microbes. In one embodiment the composition comprises from about 0.001% to about 10%, alternatively from about 0.01% to about 5%, and alternatively from about 0.05% to about 2% of an antimicrobial and/or antifungal active. Non-limiting examples of suitable antimicrobial and antifungal actives are disclosed in U.S. Pat. No. 6,607,737, issued to Bekele, et al.

8. Other Skin Care Actives

The composition of the present invention further may comprise non-vitamin antioxidant radical scavengers; preservatives; phytosterols and/or plant hormones (e.g., sitosterol, stigmasterol, campesterol, brassicasterol, kinetin, zeatin); protease inhibitors (e.g., hexamidine, vanillin acetate, menthyl anthranilate); tyrosinase inhibitors (e.g., sinablanca (mustard seed extract), tetrahydrocurcumin, cetyl pyridinium chloride); anti-inflammatory agents (e.g., glycyrrhizic acid and glycyrrhetenic acid); topical anesthetics, anti-cellulite agents, sunless tanning agents (e.g., dihydroxyacetone) and N-acyl amino acid compounds (e.g., N-undecylenoyl-L-phenylalanine, commercially available under the tradename SEPIWHITE®), and mixtures thereof.

Suitable non-vitamin antioxidant radical scavengers include, but are not limited to, BHT (butylated hydroxy toluene), L-ergothioneine (available as THIOTANE™); tetrahydrocurcumin, cetyl pyridinium chloride, carnosine, diethylhexyl syrinylidene malonate (available as OXYNEX™), ubiquinone (co-enzyme Q10), hydroxy tyrosol, butylated hydroxy benzoic acids and their salts, 6-hydroxy-2,5,7,8-tetramethylchroman-2-carboxylic acid (commercially available under the tradename TROLOX™), gallic acid and its alkyl esters, especially propyl gallate, uric acid and its salts and alkyl esters, sorbic acid and its salts, lipoic acid, amines (e.g., N,N-diethylhydroxylamine, amino-guanidine), sulfhydryl compounds (e.g., glutathione), dihydroxy fumaric acid and its salts, lycine pidolate, arginine pilolate, nordihydroguaiaretic acid, lysine, methionine, proline, superoxide dismutase, silymarin, tea extracts, grape skin and/or seed extracts, melanin, and rosemary extracts, and combinations thereof.

Other useful skin care actives include dehydroepiandrosterone (DHEA), its analogs and derivatives; alpha- and beta-hydroxyacids, including glycolic acid and octanoyl salicylate, arbutin, dimethyl aminoethanol (DMAE), kojic acid, dihydroxy acetone (DHA), soy proteins and peptides (for example, protease inhibitors such as soybean trypsin inhibitor, and Bowman-Birk inhibitor), arbutin, their isomers, salts, and derivatives, and mixtures thereof.

B. Surfactants

The composition of the present invention may include one or more surfactants. These surfactants or combinations of surfactants should be mild, which means that these surfactants provide sufficient cleansing or detersive benefits but do not overly dry the skin. Surfactants useful herein include those selected from the group consisting of anionic surfactants, amphoteric surfactants, zwitterionic surfactants, cationic surfactants, nonionic surfactants and mixtures thereof. Examples of such surfactants are found in U.S. Pat. No. 5,624,666, issued to Coffindaffer, et al. Anionic, nonionic, and cationic surfactants useful in the composition of the present invention are disclosed in McCutcheon's, Detergents and Emulsifiers, North American edition (1986), published by Allured Publishing Corporation; McCutcheon's, Functional Materials, North American Edition (1992); and U.S. Pat. No. 3,929,678, issued to Laughlin, et al. Non-limiting examples of suitable zwitterionic or amphoteric surfactants are described in U.S. Pat. No. 5,104,646 and U.S. Pat. No. 5,106,609, both issued to Bolich, Jr., et al. Concentrations of these surfactant are from about 0.1% to about 20%, alternatively from about 0.5% to about 15%, and alternatively from about 1% to about 10%.

C. Particulate Materials

The composition of the present invention may comprise a particulate material. In one embodiment, the composition may comprise from about 0.1% to about 10% of a particulate material, and alternatively from about 1% to about 5% of a particulate material. Non-limiting examples of suitable particulate materials can be found in The Cosmetic, Toiletry, and Fragrance Association's The International Cosmetic Ingredient Dictionary and Handbook, 10^{th} Ed., Gottschalck, T. E. and McEwen, Jr., Eds. (2004), p. 2728. Other suitable particulate materials include those disclosed in U.S. Patent Publication No. US2005/0214332A1, published Sep. 29, 2005.

Other examples of particulate materials useful in the present invention include colored and uncolored pigments, interference pigments, inorganic powders and organic powders other than those described above, composite powders, optical brightener particles, and mixtures thereof. The average size of such particulates in general may be smaller than the aforementioned particulate materials, ranging for example from about 0.1 microns to about 100 microns. These particulates can, for example, be platelet shaped, spherical, elongated or needle-shaped, or irregularly shaped, surface coated or uncoated, porous or non-porous, charged or uncharged, and can be added to the current composition as a powder or as a pre-dispersion. These particulate materials can be derived from natural and/or synthetic sources.

Suitable organic powders particulate materials include, but are. not limited, to spherical polymeric particles chosen from the methylsilsesquioxane resin microspheres, for example, Tospearl™ 145A, (Toshiba Silicone); microspheres of polymethylmethacrylates, for example, Micropearl™ M 100 (Seppic); the spherical particles of crosslinked polydimethylsiloxanes, for example, Trefil™ E 506C or Trefil™ E 505C (Dow Coming Toray Silicone); sphericle particles of polyamide, for example, nylon-12, and Orgasol™ 2002D Nat C05 (Atochem); polystyrene microspheres, for example Dyno Particles, sold under the name Dynospheres™, and ethylene acrylate copolymer, sold under the name FloBead™ EA209 (Kobo); aluminium starch octenylsuccinate, for example Dry Flo™ (National Starch); microspheres of polyethylene, for example Microthene™ FN510-00 (Equistar), silicone resin, polymethylsilsesquioxane silicone polymer, platelet shaped powder made from L-lauroyl lysine, and mixtures thereof.

Also useful herein are interference pigments. Intereference pigments are disclosed in U.S. Patent Publication No. US2005/0220828A1, published Oct. 6, 2005, and are available commercially from a wide variety of suppliers, for example, Rona (Timiron™ and Dichrona™), Presperse (Flonac™), Englehard (Duochrome™), Kobo (SK-45-R and SK-45-G), BASF (Sicopearls™) and Eckart (Prestige™). In one embodiment, the average diameter of the longest side of the individual particles of interference pigments is less than about 75 microns, and alternatively less than about 50 microns.

Other pigments useful in the present invention can provide color primarily through selective absorption of specific wavelengths of visible light, and include inorganic pigments, organic pigments and combinations thereof. Examples of such organic and inorganic pigments are disclosed in U.S. Patent Publication No. US2005/0220828A1, published Oct. 6, 2005. Also suitable are charged dispersions of titanium dioxide, disclosed in U.S. Pat. No. 5,997,887, issued to Ha, et al.

D. Conditioning Agents

The composition of the present invention may comprise from about 0.1% to about 50%, alternatively from about 0.5% to about 30%, alternatively from about 1% to about 20%, alternatively from about 2% to 15%, of a conditioning agent. These conditioning agents include, but are not limited to, hydrocarbon oils and waxes, emollients, silicones, fatty acid derivatives, cholesterol, cholesterol derivatives, diglycerides, triglycerides, vegetable oils, vegetable oil derivatives, acetoglyceride esters, alkyl esters, alkenyl esters, lanolin, wax esters, beeswax derivatives, sterols and phospholipids, salts, isomers and derivatives thereof, and combinations thereof. Suitable conditioning agents are exemplified in U.S. Pat. No. 5,997,890, issued to Sine, et al.

Non-limiting examples of silicone oils suitable for use herein include dimethicone copolyol, dimethylpolysiloxane, diethylpolysiloxane, mixed C₁₋₃₀ alkyl polysiloxanes, phenyl dimethicone, dimethiconol, and combinations thereof. In one embodiment, the silicone oils are non-volatile silicone oils selected from the group consisting of dimethicone, dimethiconol, mixed C₁₋₃₀ alkyl polysiloxanes, silicone crosspolymers, and combinations thereof. These and other examples of silicone oils useful herein are described in U.S. Pat. No. 5,011,681, issued to Ciotti, et al.

Non-limiting examples of silicone cross-polymers suitable for use herein include acrylate/bis-hydroxypropyl dimethicone crosspolymer, C₃₀₋₄₅ alkyl cetearyl dimethicone crosspolymer, acrylate/bis-hydroxypropyl dimethicone crosspolymer, C₃₀₋₄₅ alkyl cetearyl dimethicone crosspolymer, cetearyl dimethicone/vinyl dimethicone crosspolymer, dimethicone crosspolymer, dimethicone crosspolymer-3, dimethicone/phenyl vinyl dimethicone crosspolymer, dimethicone/vinyl dimethicone crosspolymer, diphenyl dimethicone crosspolymer, divinyldimethicone/dimethicone crosspolymer, polyethylene glycol (PEG)-10 dimethicone crosspolymer, PEG-12 dimethicone crosspolymer, PEG-10 dimethicone/vinyl dimethicone crosspolymer, PEG-10/lauryl dimethicone crosspolymer, PEG-15/lauryl dimethicone crosspolymer, trifluoropropyl dimethicone/trifluoropropyl divinyldimethicone crosspolymer, vinyl dimethicone/lauryl dimethicone crosspolymer, vinyidimethyl/trimethylsiloxysilicate stearyl dimethicone crosspolymer, polysilicone-11, and mixtures thereof.

Also useful herein are various C₁₋₃₀ monoesters and polyesters of sugars and related materials, for example, sucrose esters of fatty acids (SEFA).

E. Structuring Agent

The composition of the present invention may contain a structuring agent. Structuring agents are especially preferred in the emulsions of the present invention, and still more preferred in the oil-in-water emulsions of the present invention. Without being limited by theory, it is believed that the structuring agent assists in providing rheological characteristics (for example yield and structural characteristics) to the composition which contribute to the stability of the composition. The composition of the present invention comprise from about 0.1% to about 20%, alternatively from about 0.5% to about 10%, and alternatively from about 1% to about 5%, of one or more structuring agents. In one embodiment, structuring agents have a hydrophilic lipophilic balance (HLB) of from about 1 to about 8 and have a melting point of at least about 45° C. Non-limiting examples of suitable structuring agents are disclosed in U.S. Pat. No. 6,013,270, issued to Hargraves, et al.

F. Thickening Agent

The composition of the present invention may comprise one or more thickening agents. Herein, “thickening agent” is understood to include both thickening agents and gelling agents. Non-limiting examples of classes of suitable thickening agents include carboxylic acid polymers, crosslinked polyacrylate polymers, polyacrylamide polymers, polysaccharaides and gums, as disclosed in U.S. Patent Publication No. US2005/0214332A1, published Sep. 29, 2005, cationic polymer thickening agents, silicone elastomer polymers, and combinations thereof. In one embodiment, the cationic polymer is a polyquaternium polymer.

In one embodiment, the composition may comprise from about 0.1% to about 30%, alternatively from about 0.1% to about 20%, and alternatively from about 0.2% to about 10% of one or more thickening agents.

Non-limiting examples of useful polyquatemium polymers include, but are not limited to, acrylate/aminoacrylate/C10-30 alkyl PEG-20 itaconate copolymers such as Structure Plus™ (National Starch, Bridgewater, N.J.); and Polyquatemium-37, (methacryloylethyl trimethyl ammonium chloride homopolymer), commercially available from 3V Inc. (Weehawken, N.J.) as Synthalen™-CU, CR and CN. Polymer mixtures containing Polyquaternium-37 are also available from Ciba™ (High Point, N.C.) as Salcare™-SC95 and SC96.

When the composition of the present invention is in the form of a water-in-silicone emulsion, the composition may comprise silicone elastomer thickeners. The silicone elastomers may comprise emulsifying crosslinked siloxane elastomers, non-emulsifying crosslinked siloxane elastomers, and mixtures thereof. Non-limiting examples of suitable crosslinked organopolysiloxane elastomers are described in U.S. Pat. Nos. 5,412,004, 5,837,793, 5,811,487 4,970,252, 5,760,116, and 5,654,362. Additional crosslinked organopolysiloxane elastomers useful in the present invention are disclosed in Japanese Patent Application JP 61-18708, assigned to Pola Kasei Kogyo KK. A non-limiting example of an emulsifying elastomer comprising dimethicone copolyol crosspolymer and dimethicone is KSG-21, available from Shin EtSu™. Non-limiting example of non-emulsifying elastomers include dimethicone/vinyl dimethicone crosspolymers, for example, DC 9040 and DC 9041, available from Dow Corning™; SFE 839, available from General Electric™; KSG-15, KSG-16, and KSG-18 (dimethicone/phenyl vinyl dimethicone crosspolymer), available from Shin Etsu™; and GRANSL™ elastomers, available from Grant Industries™.

G. Substrates

The composition of the present invention may be releasably applied to a substrate material and subsequently applied to the keratinous tissue. In one embodiment, the composition is pre-combined with or deposited onto the substrate to form a wipe product, one non-limiting example of which includes disposable wipe products. Herein, “wipe product” means a substrate and a composition of the present invention which are pre-combined for later use. Wipe products may be packaged in a relatively dry state and wetted prior to use, or may be packaged having already been wetted.

Suitable wipe substrates include, but are not limited to, nonwovens, films, foams, sponges, and combinations thereof. In one embodiment, wipe substrates comprise a porous material which is capable of holding the composition within the pores of the substrate. In one embodiment, the substrate is nonwoven.

Alternatively, the substrate may be in the form of a patch and/or a mask, which may facilitate intensive treatment of selected areas of keratinous tissue, including, but not limited to, facial crows feet areas, frown lines, under eye area, etc. The patch can be occlusive, semi-occlusive or non-occlusive. The dehydroacetate salt composition can be contained within the patch or be applied to the skin prior to application of the patch. The patch can also include additional actives such as chemical initiators for exothermic reactions such as those described in PCT Application No. WO 9701313 to Burkett, et al. The patch can also contain a source of energy (e.g., a battery), for example to increase delivery of the dehydroacetate salt and other active agents.

IV. Methods of Use

The present invention provides for a method for regulating the condition of mammalian keratinous tissue, comprising the step of topically applying to mammalian keratinous tissue an effective amount of a personal care composition of the present invention. Alternatively, the composition is applied to mammalian skin. Alternatively, the composition is applied to human skin. A wide range of quantities of the composition of the present invention can be employed to improve the condition of the skin. Quantities of the present composition typically applied per cm² of skin are from about 0.1 mg/cm² to about 20 mg/cm². Alternatively, a suitable application amount is about 0.5 mg/cm² to about 10 mg/cm². The composition may be applied to any part of the external portion of keratinous tissue. In one embodiment, the composition is delivered to the face and/or neck. The amount of the composition applied, the frequency of application and the period of use will vary widely depending upon the level of components of a given composition and the level of regulation desired. In one embodiment, the composition is applied at least once daily, where “daily” and “days” mean a 24-hour period. For example, the composition may be applied daily for 30 consecutive days, alternatively for 14 consecutive days, alternatively for 7 consecutive days, and alternatively for 2 consecutive days.

The composition of the present invention may be applied using the palms of the hands, the fingers, or by using a delivery enhancement device and/or energy delivery device. The composition may be releasably applied to a substrate. In one embodiment, a composition may be applied in the form of a lotion, cleansing milk, cream, gel, foam, ointment, paste, emulsion, tonic, cosmetic, etc. and said composition allowed to remain on the keratinous tissue for a sufficient period of time to produce some benefit (i.e., a “leave-on” composition). In an alternative embodiment, the composition may be rinsed, wiped, or otherwise removed from the keratinous tissue after application.

When the composition of the present invention is applied in combination with a patch or a mask, the patch or mask may be left on the keratinous tissue for a period of about 5 minutes, alternatively for about 15 minutes, alternatively for about 30 minutes, alternatively for about 1 hour, alternatively for about six hours, and alternatively overnight.

V. Kit

The present invention further may comprise a kit, said kit comprising a personal care composition as described herein. The kit further may comprise one or more additional compositions, instructions for applying the composition(s), instructions for complying with a suitable application regimen, an implement, a substrate, a delivery enhancement device, a dietary supplement, and combinations thereof.

The kit may comprise an outer packaging unit, which in turn may comprise one or more smaller, inner packaging units. The inner packaging units may comprise one or more of the individual components of the kit. The inner packaging units each may contain a quantity of a composition suitable for use in a single application regimen. In one example, the individual packaging units each will contain 10 ml, alternatively 5 ml, alternatively 2 ml, and alternatively 1 ml of a composition described herein.

### EXAMPLES

### Examples 1-7

A Moisturizing Skin Cream/Lotion may be Prepared from the Following Components.

| Component | | | | | | |
|---|---|---|---|---|---|---|
| 1 | 2 | 3 | 4 | 5 | 6 | 7 |
| % | % | % | % | % | % | % |

Phase A

| | | | | | | | |
|---|---|---|---|---|---|---|---|
| Water | qs | qs | qs | qs | qs | qs | qs |
| Glycerol | 5.00 | 7.00 | 7.00 | 10.00 | 5.00 | 10.00 | 5.00 |
| Phenylbenzimidazole | — | — | — | — | 1.25 | — | — |
| Sulfonic Acid | | | | | | | |
| Disodium EDTA | 0.10 | 0.10 | 0.10 | 0.10 | 0.10 | 0.10 | 0.10 |
| Allantoin | 0.20 | 0.20 | 0.20 | 0.20 | 0.20 | 0.20 | 0.20 |
| Sodium Dehydroacetate | 0.10 | 0.20 | 0.30 | 0.50 | 1.00 | 2.00 | 5.00 |
| N-acetyl glucosamine | — | 2.50 | — | 2.00 | — | — | 2.00 |
| Sodium metabisulfite | 0.10 | 0.20 | — | — | — | 0.10 | — |
| BHT | 0.015 | — | — | — | 0.015 | 0.015 | — |
| Titanium Dioxide | 0.25 | 0.45 | 0.45 | 0.75 | 0.55 | 0.45 | — |
| Dexpanthenol | 0.25 | 0.50 | 1.00 | 2.00 | 1.00 | 1.00 | 0.50 |
| Palmitoyl-pentapeptide | — | — | 0.0004 | — | 0.0003 | 0.0006 | — |

Phase B

| | | | | | | | |
|---|---|---|---|---|---|---|---|
| C12-C15 alkyl benzoate | 5.00 | 2.50 | 1.50 | 2.50 | — | 2.50 | 1.00 |
| Caprylic/Capric | 1.0 | 1.50 | 1.50 | 1.50 | 1.50 | 1.50 | — |
| Triglyceride | | | | | | | |
| Octyl Salicylate | — | — | — | — | 5.00 | — | — |
| Octocrylene | — | — | — | — | 1.00 | — | — |
| Butyl Methoxydibenzoyl- | — | — | — | — | 2.00 | — | — |
| methane | | | | | | | |
| Cetyl Alcohol | 0.50 | 0.50 | 0.50 | 0.50 | 0.50 | 0.50 | 0.40 |
| Tocopherol Acetate | — | 0.50 | 0.50 | 0.50 | 0.50 | 0.50 | 0.50 |
| Sorbitan Stearate/Sucrose | 1.00 | 1.00 | 1.00 | 1.00 | 1.00 | 1.00 | 1.00 |
| Cocoate | | | | | | | |
| Cetearyl Glucoside | 0.50 | 0.50 | 0.50 | 0.50 | 0.50 | 0.50 | 0.10 |
| Stearyl Alcohol | 0.70 | 0.70 | 0.70 | 0.70 | 0.70 | 0.70 | 0.70 |
| Behenyl Alcohol | 0.60 | 0.60 | 0.60 | 0.60 | 0.60 | 0.60 | 0.60 |
| Ethyl Paraben | 0.20 | 0.20 | 0.20 | 0.20 | 0.20 | 0.20 | 0.20 |
| Propyl Paraben | 0.10 | 0.10 | 0.10 | 0.10 | 0.10 | 0.10 | 0.10 |
| PEG-100 Stearate | 0.10 | 0.10 | 0.10 | 0.10 | 0.10 | 0.10 | 0.10 |
| Polymethylsilsesquioxane | 0.25 | 0.50 | 1.50 | 0.50 | 0.25 | 0.50 | 0.50 |

Phase C

| | | | | | | | |
|---|---|---|---|---|---|---|---|
| Polyacrylamide/C13-14 | 2.00 | 2.25 | 2.50 | 2.50 | 1.50 | 2.50 | 4.00 |
| Isoparaffin/Laureth-7 | | | | | | | |

Phase D

| | | | | | | | |
|---|---|---|---|---|---|---|---|
| Retinyl Propionate | — | 0.10 | 0.10 | 0.10 | — | — | — |
| Phytosterol | 1.00 | — | — | — | — | — | — |
| Green Tea Extract | 1.00 | 1.00 | 1.00 | 1.00 | — | — | — |
| Benzyl Alcohol | 0.25 | 0.25 | 0.25 | 0.25 | 0.25 | 0.25 | 0.25 |
| Dimethicone/Dimethi- | 0.5 | 1.00 | 2.50 | 0.2500 | 2.00 | 2.00 | 2.00 |
| conol | | | | | | | |
| Triethanolamine | Add to | Add to | Add to | Add to | Add to | Add to | Add to |
| | adjust | adjust | adjust | adjust | adjust | adjust | adjust |
| | to pH 7.0 | to pH 7.25 | to pH 7.50 | to pH 7.75 | to pH 8.00 | to pH 7.50 | to pH 7.80 |

In a suitable vessel, combine Phase A components and mix with a suitable mixer (e.g., Tekmar RW20DZM). Heat with stirring to a temperature of about 70-80° C., and maintain the temperature. In a separate suitable vessel, combine the Phase B components and mix with a suitable mixer. Heat with stirring to about 70-75° C., and maintain the temperature. Add the Phase B mixture to the Phase A mixture and mix well so as to emulsify the combination. Allow the emulsion of Phase A and B components to cool to about 60° C. and then add the Phase C components to the emulsion with continuous mixing. Cool the emulsion of Phase A, B and C components to about 40° C. Add the Phase D components with mixing to the emulsion. Add sufficient triethanolamine to attain a desired final pH while monitoring the pH with a pH meter. Mill the resulting emulsion using a suitable mill (Tekmar T-25) for about 5 minutes or until the product is uniform.

### Examples 8-13

A Moisturizing Skin Cream/Lotion may be Prepared from the Following Components.

| Component | | | | | |
|---|---|---|---|---|---|
| 8 | 9 | 10 | 11 | 12 | 13 |
| % | % | % | % | % | % |

Phase A

| | | | | | | |
|---|---|---|---|---|---|---|
| Water | qs | qs | qs | qs | qs | qs |
| Allantoin | 0.20 | 0.20 | 0.20 | 0.20 | 0.20 | 0.20 |
| Disodium EDTA | 0.10 | 0.10 | 0.10 | 0.10 | 0.10 | 0.10 |
| Ethyl Paraben | 0.20 | 0.20 | 0.20 | 0.20 | 0.20 | 0.20 |
| Propyl Paraben | 0.10 | 0.10 | 0.10 | 0.10 | 0.10 | 0.10 |
| Bht | 0.015 | 0.015 | 0.015 | 0.015 | 0.015 | — |
| Dexpanthenol | 1.00 | 0.50 | 1.00 | 1.00 | 1.00 | 0.50 |
| Glycerin | 7.50 | 10.00 | 15.00 | 7.50 | 5.00 | 10.00 |
| Sodium Dehydroacetate | 0.20 | 2.00 | 0.10 | 0.50 | 1.00 | 0.10 |
| Hexamidine Isethionate | — | 0.10 | — | — | — | 0.10 |
| Palmitoyl-Pentapeptide | — | — | — | 0.0004 | 0.0003 | — |
| Phenylbenzimidazole | — | — | — | — | 1.00 | — |
| Sulfonic Acid | | | | | | |
| Benzyl Alcohol | 0.25 | 0.25 | 0.25 | 0.25 | 0.25 | 0.25 |
| Green Tea Extract | 1.00 | 1.00 | 1.00 | 1.00 | 1.00 | 1.00 |
| N-Acetyl Glucosamine | — | 1.00 | — | — | — | 2.00 |
| Sodium Metabisulfite | 0.10 | — | — | — | 0.10 | — |
| Triethanolamine | Add to | Add to | Add to | Add to | Add to | Add to |
| | adjust | adjust | adjust | pH | adjust | adjust |
| | to pH 7.0 | to pH 7.25 | to pH 7.50 | adjust to 8.00 | to pH 7.50 | to pH 7.80 |

PHASE B

| | | | | | | |
|---|---|---|---|---|---|---|
| Cyclopentasiloxane | 15.00 | 15.00 | 18.00 | 15.00 | 15.00 | 15.00 |
| Titanium Dioxide | 0.50 | 0.50 | 0.75 | 0.50 | 0.50 | — |

PHASE C

| | | | | | | |
|---|---|---|---|---|---|---|
| C12-C15 Alkyl Benzoate | 3.00 | 0.50 | 2.00 | — | 4.50 | — |
| Vitamin E Acetate | 0.50 | — | 1.00 | 0.50 | 0.50 | 0.50 |
| Retinyl Propionate | 0.30 | 0.10 | — | 0.20 | 0.20 | — |
| Phytosterol | 2.00 | — | 1.00 | — | 3.00 | — |

PHASE D

| | | | | | | |
|---|---|---|---|---|---|---|
| KSG-21 Silicone Elastomer¹ | 4.00 | 4.00 | 5.00 | 4.00 | 4.00 | 4.00 |
| Dow Corning 9040 Silicone | 15.00 | 15.00 | 12.00 | 15.00 | 15.00 | 15.00 |
| Elastomer | | | | | | |
| Abil Em-97 Dimethicone | 0.50 | — | — | 0.50 | 0.50 | 0.50 |
| Copolyol² | | | | | | |
| Polymethylsilsesquioxane | 2.50 | 2.50 | 2.00 | 2.50 | 2.50 | 2.50 |

¹KSG-21, an emulsifying silicone elastomer available from Shin Etsu ™
²Available from Goldschmidt Chemical Corporation ™

In a suitable vessel, blend the Phase A components together with a suitable mixer (e.g., Tekmar model RW20DZM) and mix until all of the components are dissolved. Adjust the pH of Phase A by adding a sufficient amount of triethanolamine while monitoring the pH with a suitable pH meter. Blend the Phase B components together in suitable vessel and mill using a suitable mill (e.g., Tekmar RW-20) for about 5 minutes. Add the Phase C components to the Phase B mixture with mixing. Add the Phase D components to the mixture of Phases B and C. Mix the resulting combination of Phase B, C, and D components using a suitable mixer (e.g., Tekmar RW-20) for about 1 hour. Slowly add Phase A to the mixture of Phases B, C, and D with mixing. Mix the resulting mixture until the product is uniform and mill for about 5 minutes using an appropriate mill (e.g., Tekmar T-25).

### Example 14

A Moisturizing Silicone-in-Water Serum/Lotion may be Prepared from the Following Components:

Water phase: Acrylates/C10-30 alkyl acrylates crosspolymer (0.2%); Glycerin (6%); Disodium EDTA (0.1%); D-panthenol (0.5%); Hexamidine diisethionate (0.1%); Sodium Dehydroacetate (0.5%); Water (q.s).
Silicone phase: Cyclomethicone D5 (15%); Dow Corning 9040 silicone elastomer¹ (5%); Dimethicone/Dimethiconol (2%); Dimethicone 50 csk (1%); Vitamin E Acetate (0.5%).
   ¹A silicone elastomer dispersion from Dow Corning™
Thickener: Polyacrylamide/C13-14 isoparaffin/laureth-7 (2.5%).
Other Ingredients: Polyethylene (1%); Flamenco Summit Green G30D² (0.5%), Prestige Silk Red³ (0.5%); Triethanolamine (q.s. to adjust to pH 7.25).
   ²Titanium dioxide and tin oxide coated mica green interference pigment from Engelhard™
   ³Titanium dioxide coated mica red interference pigment from Eckart™

### Example 15

A Silicone-in-Water Foaming Mousse may be Prepared from the Following Components:

Water phase: Glycerin (3%); Disodium EDTA (0.1%); D-panthenol (0.5%); Sodium Dehydroacetate (0.25%); Ascorbyl Glucoside (1%); Soy Isoflavone (0.1%); N-acetyl Glucosamine (2%); Water (q.s).
Silicone phase: Cyclomethicone D5 (10%); Dow Corning 9040 silicone elastomer¹ (5%); Dimethicone/Dimethiconol (2%); Vitamin E Acetate (0.1%).
   ¹A silicone elastomer dispersion from Dow Corning™
Thickener: Polyacrylamide/C13-14 isoparaffin/laureth-7 (1.5%).
Other Ingredients: Polymethylsil-sesquioxane (0.5%); Prestige Silk Red² (1.0%); Triethanolamine (q.s. to adjust to pH 7.5). Propellants: 152A HFC (3%); A-70 (3%).
   ²Titanium dioxide coated mica red interference pigment from Eckart™

Procedure for preparing Examples 14 and 15: In a suitable vessel, combine the water phase ingredients and mix until uniform. In a separate suitable container, combine the silicone/oil phase ingredients and mix until uniform. Add the silicone/oil phase to the water phase and mill the resulting emulsion (e.g., with a Tekmar T-25). Add the thickener and then the remaining ingredients to the emulsion while stirring. The amount of triethanolamine to be added is based on desired final pH and is added while monitoring the pH with a pH meter. For Example 14, when the composition is uniform, pour the product into one or more suitable containers. For Example 15, when the composition is uniform, pour the product and propellant into one or more suitable aerosol containers prior to sealing the container.

### Example 16

Example 16 Exemplifies a Water-Based Stick Formulation.

The following ingredients are combined: 15% propylene glycol; 50% dipropylene glycol, 6% sodium stearate, 2% N-acetyl-D-glucosamine, 2.5% sodium dehydroacetate, water (q.s.). Adjust the resulting composition to a pH of about 8.0 by adding triethanolamine while monitoring with a pH meter. Mix all ingredients thoroughly and combine into one or more appropriately size containers. Heat to approximately 85° C., cool, and pour into appropriate stick containers at approximately 65° C.

The dimensions and values disclosed herein are not to be understood as being strictly limited to the exact numerical values recited. Instead, unless otherwise specified, each such dimension is intended to mean both the recited value and a functionally equivalent range surrounding that value. For example, a dimension disclosed as “40 mm” is intended to mean “about 40 mm”.

All documents cited in the Detailed Description of the Invention are, in relevant part, incorporated herein by reference; the citation of any document is not to be construed as an admission that it is prior art with respect to the present invention. To the extent that any meaning or definition of a term in this written document conflicts with any meaning or definition of the term in a document incorporated by reference, the meaning or definition assigned to the term in this written document shall govern.

While particular embodiments of the present invention have been illustrated and described, it would be obvious to those skilled in the art that various other changes and modifications can be made without departing from the spirit and scope of the invention. It is therefore intended to cover in the appended claims all such changes and modifications that are within the scope of this invention.

What is claimed is:

1. A personal care composition comprising:
a) a dehydroacetate salt; and
b) a dermatologically acceptable carrier;
wherein the pH of said composition is about 7.0 and greater.

2. The composition of claim 1, wherein said dehydroacetate salt is selected from the group consisting of sodium dehydroacetate, potassium dehydroacetate, triethanolamine dehydroacetate, and mixtures thereof.

3. The composition of claim 2, wherein said dehydroacetate salt is sodium dehydroacetate.

4. The composition of claim 1, wherein the composition comprises from about 0.001% to about 10% of the dehydroacetate salt.

5. The composition of claim 1, wherein the pH of said composition is from about 7.0 to about 11.0.

6. The composition of claim 5, wherein the pH of said composition is from about 7.0 to about 9.0.

7. The composition of claim 1, further comprising a particulate material.

8. The composition of claim 1, further comprising at least one additional skin care active.

9. The composition of claim 8, wherein said additional skin care active is selected from the group consisting of vitamin B compounds, vitamin E compounds, vitamin C compounds, vitamin D compounds, peptides, sugar amines, protease inhibitors, sunscreens, desquamation agents, chelators, skin lightening compounds, non-vitamin antioxidant radical scavengers, phytosterols, plant hormones, protease inhibitors, tyrosinase inhibitors, anti-inflammatory agents, topical anesthetics, anti-cellulite agents, sunless tanning agents, N-acyl amino acid compounds and derivatives, and mixtures thereof.

10. The composition of claim 9, wherein the additional skin care active is selected from the group consisting of tocopheryl nicotinate, N-acetyl-D-glucosamine, hexamidine, ascorbyl glucoside, palmitoyl-lysine-threonine-threonine-lysine-serine, butylated hydroxytoluene (BHT), cetyl betaine, bisabolol, green tea extract, theophylline, undecenoyl phenylalanine, cetyl pyridinium chloride, dihydroxyacetone, panthenol, butyl methoxydibenzoylmethane, derivatives and mixtures thereof.

11. A method of regulating the condition of mammalian keratinous tissue, comprising the step of applying to the keratinous tissue a personal care composition comprising:
a) a dehydroacetate salt, and
b) a dermatologically acceptable carrier;
wherein the pH of said composition is about 7.0 and greater.

12. The method of claim 11, wherein said personal care composition comprises from about 0.001% to about 10% of sodium dehydroacetate.

13. The method of claim 11, wherein said personal care composition further comprises at least one additional skin care active selected from the group consisting of vitamin B compounds, vitamin E compounds, vitamin C compounds, vitamin D compounds, peptides, sugar amines, protease inhibitors, and derivatives, and mixtures thereof.

14. The method of claim 13, wherein the additional skin care active is selected from the group consisting of tocopheryl nicotinate, N-acetyl-D glucosamine, hexamidine, ascorbyl glucoside, palmitoyl-lysine-threonine-threonine-lysine-serine, and mixtures thereof.

15. The method of claim 11, wherein said personal care composition is deposited onto a substrate.

16. A kit comprising:
a) a personal care composition comprising a dehydroacetate salt and a dermatologically acceptable carrier, wherein the pH of the composition is about 7.0 and greater; and
b) at least one additional personal care composition.

17. The kit of claim 16, wherein said additional composition is a dietary supplement.

18. The kit of claim 16, further comprising an implement.

19. The kit of claim 16, further comprising a delivery enhancement device.

20. The kit of claim 16, further comprising instructions for complying with an application regimen.
